(19) United States
(12) Patent Application Publication
(10) Pub. No.: US 2012/0214190 A1
(43) Pub. Date: Aug. 23, 2012

(54) System and method based on blood components for estimating human physiological parameters

(76) Inventor: Wanqiu Hou, Los Angeles, CA (US)

(21) Appl. No.: 13/400,443

(22) Filed: Feb. 20, 2012

(51) Int. Cl. C12Q 1/02; C12N 5/0786; C12M 1/34; C07K 14/525

(52) U.S. Cl. 435/29; 530/351; 435/325; 435/288.7

(57) ABSTRACT

A system and method based on blood components for estimating human general physiological parameters comprises determining the predetermined value of general physiological parameters indicative of illness and disease to monitor health status at different time points on the basis of basic physiological parameters indicated as the percentage of a particular cytokine-producing cells in a type of blood cells in a human peripheral blood sample without in vitro culture and with two-stage in vitro cultures in the absence and presence of microbial stimulation after being taken from the subject.

### CROSS-REFERENCE TO RELATED APPLICATIONS

No

### BACKGROUND-PRIOR ART

(the following is a tabulation of some prior art that presently appears relevant):
Geissmann F et al. Development of monocytes, macrophages, and dendritic cells, Science 2010 327:656-661.
Locksley R M et al. The TNF and TNF receptor superfamilies: integrating mammalian biology, Cell 2001 104:487-501.
Feldmann M and Maini R N. Anti-TNF therapy, from rationale to standard of care: what lessons has it taught us? J Immunol 2010 185:791-794.

### DESCRIPTION

1. Field of the Invention

The present invention relates to a system and method based on blood components for estimating human physiological parameters to monitor human health status. These blood components are TNF-producing monocytes in total of monocytes in a human peripheral blood sample without in vitro culture and with two-stage in vitro cultures in the absence and presence of microbial stimulation after being taken from the subject.

2. Background of the Invention

Human health status varies by individuals and is even dynamic in same individual at different environmental conditions. Although biotechnology has been acknowledged for more than 100 years, actively monitoring human health status with physiological parameters to ultimately avoid the development of illness and diseases remains a clinical and laboratory challenge.

Monocytes reside in bone marrow, blood as well as spleen and play multiple roles in immune function, for example, ingesting foreign or abnormal cells and toxic molecules, replenishing resident macrophages and dendritic cells in normal and pathological situation, and producing inflammatory mediators (Geissmann et al, 2010, Science, 327:656-661).

TNF is a cytokine involved in systemic inflammation and cause the acute phase reaction (Locksley et al, 2001 Cell, 104:487-501; Feldmann and Maini, 2010, J Immunol, 185:791-794). TNF dysregulation occurs in a variety of human diseases including autoimmune diseases, inflammatory diseases, bacterial, viral and parasitic infections, cancer, major depression disorder and neurodegenerative diseases.

Monocytes have a strong potential to produce TNF in response to self and non-self stimuli (Geissmann et al, 2010, Science, 327:656-661). Monitoring TNF-producing monocytes in total of monocytes in a human blood sample without in vitro culture and with two-stage in vitro cultures in the absence and presence of microbial stimulation after being taken from the subject and thereby generating human physiological parameters based on this monitoring by using biotechnology, computer and network technology may be very helpful to estimate human health status and avoid illness and disease development.

### SUMMARY OF THE INVENTION

The present invention provides a system and method based on blood components for estimating human general physiological parameters comprising determining the predetermined value of general physiological parameters indicative of illness and disease to monitor health status at different time points on the basis of basic physiological parameters indicated as the percentage of a particular cytokine-producing cells in a type of blood cells in a human peripheral blood sample without in vitro culture and with two-stage in vitro cultures in the absence and presence of microbial stimulation after being taken from the subject.

In one aspect, a system and method based on blood components for estimating human general physiological parameters comprises determining the predetermined value of general physiological parameters from basic physiological parameters indicated as the percentage of a particular cytokine-producing cells in a type of blood cells in a human peripheral blood sample without in vitro culture and with two-stage in vitro cultures after being taken from the subject.
1. As used herein, the term “a particular cytokine” refers to TNF,
2. As used herein, the term “a type of blood cells” refers to monocyte,
3. As used herein, the term “two-stage” refers to 1 hour and 4 hours,
4. As used herein, the term “the percentage of a particular cytokine-producing cells in a type of blood” refers to the percentage of TNF-producing monocytes in total of monocytes,
5. As used herein, the term “basic physiological parameters” refers to the percentage of TNF-producing monocytes in total of monocytes without in vitro culture, the percentage of TNF-producing monocytes in total of monocytes with 1 hour in vitro culture, and the percentage of TNF-producing monocytes in total of monocytes with 4 hours in vitro culture,
6. As used herein, the term “value of general physiological parameters” refers to calculated data by dividing the percentage of TNF-producing monocytes in total of monocytes with 1 hour in vitro culture by the percentage of TNF-producing monocytes in total of monocytes with 4 hours in vitro culture.

In another aspect, a system and method based on blood components for estimating human stimulatory general physiological parameters comprises determining the predetermined value of stimulatory general physiological parameters from basic physiological parameters indicated as the percentage of a particular cytokine-producing cells following microbial stimulation in a type of blood cells in a human peripheral blood sample without in vitro culture and with two-stage in vitro cultures after being taken from the subject.
1. As used herein, the term “a particular cytokine” refers to TNF,
2. As used herein, the term “a type of blood cells” refers to monocyte,
3. As used herein, the term “two-stage” refers to 1 hour and 4 hours,
4. As used herein, the term “microbial stimulation” refers to influenza A virus at a multiplicity of infection 10 per cell,
5. As used herein, the term “the percentage of a particular cytokine-producing cells in a type of blood” refers to the percentage of TNF-producing monocytes in total of monocytes,
6. As used herein, the term “basic physiological parameters” refers to the percentage of TNF-producing monocytes in total of monocytes following influenza A virus at a multiplicity of infection 10 per cell without in vitro culture, the percentage of TNF-producing monocytes in total of monocytes following influenza A virus at a multiplicity of infection 10 per cell with 1 hour in vitro culture, and the percentage of TNF-producing monocytes in total of monocytes following influenza A virus at a multiplicity of infection 10 per cell with 4 hours in vitro culture,
7. As used herein, the term “value of stimulatory general physiological parameters” refers to calculated data by dividing the percentage of TNF-producing monocytes in total of monocytes following influenza A virus at a multiplicity of infection 10 per cell with 1 hour in vitro culture by the percentage of TNF-producing monocytes in total of monocytes following influenza A virus at a multiplicity of infection 10 per cell with 4 hours in vitro culture.

In another aspect, a system and method based on blood components for estimating human relative general physiological parameters comprises determining the predetermined value of relative general physiological parameters from basic physiological parameters indicated as the percentage of a particular cytokine-producing cells following either no or microbial stimulation in a type of blood cells in a human peripheral blood sample without in vitro culture and with two-stage in vitro cultures after being taken from the subject.
1. As used herein, the term “a particular cytokine” refers to TNF,
2. As used herein, the term “a type of blood cells” refers to monocyte,
3. As used herein, the term “two-stage” refers to 1 hour and 4 hours,
4. As used herein, the term “microbial stimulation” refers to influenza A virus at a multiplicity of infection 10 per cell,
5. As used herein, the term “the percentage of a particular cytokine-producing cells in a type of blood” refers to the percentage of TNF-producing monocytes in total of monocytes,
6. As used herein, the term “basic physiological parameters” refers to the percentage of TNF-producing monocytes in total of monocytes following no stimulation without in vitro culture, the percentage of TNF-producing monocytes in total of monocytes following influenza A virus at a multiplicity of infection 10 per cell without in vitro culture, the percentage of TNF-producing monocytes in total of monocytes following no stimulation with 1 hour in vitro culture, the percentage of TNF-producing monocytes in total of monocytes following influenza A virus at a multiplicity of infection 10 per cell with 1 hour in vitro culture, the percentage of TNF-producing monocytes in total of monocytes following no stimulation with 4 hours in vitro culture, and the percentage of TNF-producing monocytes in total of monocytes following influenza A virus at a multiplicity of infection 10 per cell with 4 hours in vitro culture,
7. As used herein, the term “value of relative general physiological parameters” refers to calculated data by dividing the percentage of TNF-producing monocytes in total of monocytes following no stimulation with 4 hours in vitro culture by the percentage of TNF-producing monocytes in total of monocytes following influenza A virus at a multiplicity of infection 10 per cell with 4 hours in vitro culture.

In another yet aspect, a system and method based on blood components for estimating human physiological parameters comprises 10-ml syringe with an 18-G needle for blood collection, 15-ml polystyrene conical centrifuge tube and 5-ml polystyrene round bottom test tube for preparing, handling and storing blood cells, CO2 incubator for blood cell culture, hemocytometer and light microscope for counting blood cell number, BD LSR II flow cytometer for counting and examining a particular cytokine-producing cells in a type of blood cells, data acquisition network terminal composing of computers for acquiring sample data from BD LSR II flow cytometer, storing sample data, exporting sample data in the Flow Cytometry Standard format with an .fcs file extension and sending exported data to blood analysis server with a secure and agreed format through intranet or internet, and blood analysis server composing of computer database server for analyzing exported data from data acquisition network terminal with a secure and agreed format through intranet or internet, generating human physiological parameters, comparing physiological parameter record of the subject over time, presenting data report to the subject with physical and electronic documents and maintaining the database of the subject.

The present invention relates to a system and method based on blood components for estimating human physiological parameters to monitor human health status at different time points. These physiological parameters are useful for providing the kinetics of human health status and preventing illness and diseases.

### BRIEF DESCRIPTION OF THE DRAWINGS

FIG. 1 shows an optimized model of the system based on blood components for estimating human physiological parameters.

### DETAILED DESCRIPTION OF THE INVENTION

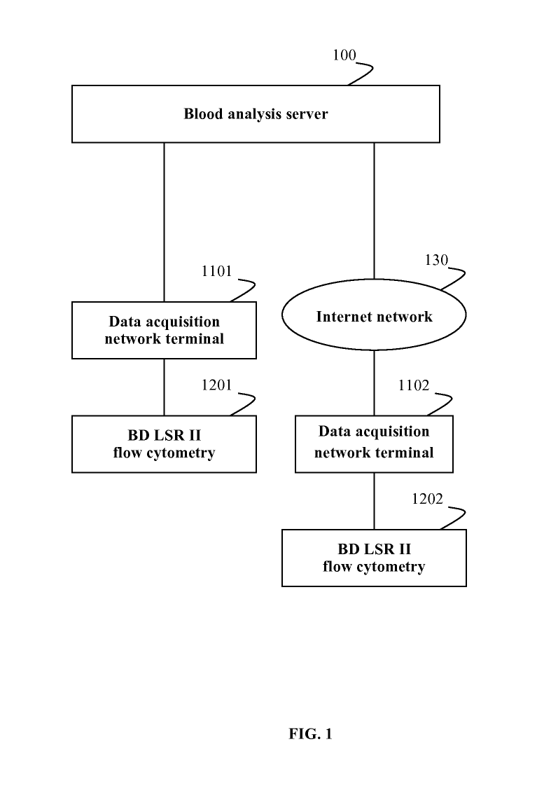

A system and method based on blood components for estimating human physiological parameters are shown in FIG. 1.

Through internet network 130 by established logical link connection with TCP/IP socket, blood analysis server 100 acquires the data for blood analysis from data acquisition network 1102 with a secure and agreed format of data files.

Through intranet network by established logical link connection with TCP/IP socket, blood analysis server 100 acquires the data for blood analysis from data acquisition network 1101 with a secure and agreed format of data files.

Data acquisition network 1101 composing of the computer running BD FACSDiva software is connected with BD LSR II flow cytometer 1201 via a computer I/O.

Data acquisition network 1102 composing of the computer running BD FACSDiva software is connected with BD LSR II flow cytometer 1202 via a computer I/O.

BD LSR II flow cytometer 1201 is an equipment for counting and examining blood components.

BD LSR II flow cytometer 1202 is an equipment for counting and examining blood components.

A system and method based on blood components for estimating human physiological parameters comprises the following items:
1. 10-ml syringe with an 18-G needle for blood collection,
2. 15-ml polystyrene conical centrifuge tube and 5-ml polystyrene round bottom test tube for preparing, handling and storing blood cells,
3. CO2 incubator for blood cell culture,
4. hemocytometer and light microscope for counting blood cell number,
5. BD LSR II flow cytometer 1201 for counting and examining a particular cytokine-producing cells in a type of blood cells,
6. BD LSR II flow cytometer 1202 for counting and examining a particular cytokine-producing cells in a type of blood cells,
7. Data acquisition network terminal 1101 composing of computers for acquiring sample data from BD LSR II flow cytometer 1201, storing sample data, exporting sample data in the Flow Cytometry Standard format with an .fcs file extension, and sending exported data to blood analysis server 100 with a secure and agreed format through intranet,
8. Blood analysis server 100 composing of computer database server for analyzing exported data from data acquisition network terminal 1101 with a secure and agreed format through intranet, generating human physiological parameters, comparing physiological parameter record of the subject over time, presenting data report to the subject with physical and electronic documents, and maintaining the database of the subject,
9. Data acquisition network terminal 1102 composing of computers for acquiring sample data from BD LSR II flow cytometer 1202, storing sample data, exporting sample data in the Flow Cytometry Standard format with an .fcs file extension, and sending exported data to blood analysis server 100 with a secure and agreed format through internet network 130,
10. Blood analysis server 100 composing of computer database server for analyzing exported data from data acquisition network terminal 1102 with a secure and agreed format through internet network 130, generating human physiological parameters, comparing physiological parameter record of the subject over time, presenting data report to the subject with physical and electronic documents, and maintaining the database of the subject.

A system and method based on blood components for estimating human general physiological parameters comprises determining the predetermined value of general physiological parameters from basic physiological parameters indicated as the percentage of a particular cytokine-producing cells in a type of blood cells in a human peripheral blood sample without in vitro culture and with two-stage in vitro cultures after being taken from the subject.
1. As used herein, the term “a particular cytokine” refers to TNF,
2. As used herein, the term “a type of blood cells” refers to monocyte,
3. As used herein, the term “two-stage” refers to 1 hour and 4 hours,
4. As used herein, the term “the percentage of a particular cytokine-producing cells in a type of blood” refers to the percentage of TNF-producing monocytes in total of monocytes,
5. As used herein, the term “basic physiological parameters” refers to the percentage of TNF-producing monocytes in total of monocytes without in vitro culture, the percentage of TNF-producing monocytes in total of monocytes with 1 hour in vitro culture, and the percentage of TNF-producing monocytes in total of monocytes with 4 hours in vitro culture,
6. As used herein, the term “value of general physiological parameters” refers to calculated data by dividing the percentage of TNF-producing monocytes in total of monocytes with 1 hour in vitro culture by the percentage of TNF-producing monocytes in total of monocytes with 4 hours in vitro culture.

A system and method based on blood components for estimating human stimulatory general physiological parameters comprises determining the predetermined value of stimulatory general physiological parameters from basic physiological parameters indicated as the percentage of a particular cytokine-producing cells following microbial stimulation in a type of blood cells in a human peripheral blood sample without in vitro culture and with two-stage in vitro cultures after being taken from the subject.
1. As used herein, the term “a particular cytokine” refers to TNF,
2. As used herein, the term “a type of blood cells” refers to monocyte,
3. As used herein, the term “two-stage” refers to 1 hour and 4 hours,
4. As used herein, the term “microbial stimulation” refers to influenza A virus at a multiplicity of infection 10 per cell,
5. As used herein, the term “the percentage of a particular cytokine-producing cells in a type of blood” refers to the percentage of TNF-producing monocytes in total of monocytes,
6. As used herein, the term “basic physiological parameters” refers to the percentage of TNF-producing monocytes in total of monocytes following influenza A virus at a multiplicity of infection 10 per cell without in vitro culture, the percentage of TNF-producing monocytes in total of monocytes following influenza A virus at multiple of infection 10 with 1 hour in vitro culture, and the percentage of TNF-producing monocytes in total of monocytes following influenza A virus at a multiplicity of infection 10 per cell with 4 hours in vitro culture,
7. As used herein, the term “value of stimulatory general physiological parameters” refers to calculated data by dividing the percentage of TNF-producing monocytes in total of monocytes following influenza A virus at a multiplicity of infection 10 per cell with 1 hour in vitro culture by the percentage of TNF-producing monocytes in total of monocytes following influenza A virus at a multiplicity of infection 10 per cell with 4 hours in vitro culture.

A system and method based on blood components for estimating human relative general physiological parameters comprises determining the predetermined value of relative general physiological parameters from basic physiological parameters indicated as the percentage of a particular cytokine-producing cells following either no or microbial stimulation in a type of blood cells in a human peripheral blood sample without in vitro culture and with two-stage in vitro cultures after being taken from the subject.
1. As used herein, the term “a particular cytokine” refers to TNF,
2. As used herein, the term “a type of blood cells” refers to monocyte,
3. As used herein, the term “two-stage” refers to 1 hour and 4 hours,
4. As used herein, the term “microbial stimulation” refers to influenza A virus at a multiplicity of infection 10 per cell,
5. As used herein, the term “the percentage of a particular cytokine-producing cells in a type of blood” refers to the percentage of TNF-producing monocytes in total of monocytes,
6. As used herein, the term “basic physiological parameters” refers to the percentage of TNF-producing monocytes in total of monocytes following no stimulation without in vitro culture, the percentage of TNF-producing monocytes in total of monocytes following influenza A virus at a multiplicity of infection 10 per cell without in vitro culture, the percentage of TNF-producing monocytes in total of monocytes following no stimulation with 1 hour in vitro culture, the percentage of TNF-producing monocytes in total of monocytes following influenza A virus at a multiplicity of infection 10 per cell with 1 hour in vitro culture, the percentage of TNF-producing monocytes in total of monocytes following no stimulation with 4 hours in vitro culture, and the percentage of TNF-producing monocytes in total of monocytes following influenza A virus at a multiplicity of infection 10 per cell with 4 hours in vitro culture,
7. As used herein, the term “value of relative general physiological parameters” refers to calculated data by dividing the percentage of TNF-producing monocytes in total of monocytes following no stimulation with 4 hours in vitro culture by the percentage of TNF-producing monocytes in total of monocytes following influenza A virus at a multiplicity of infection 10 per cell with 4 hours in vitro culture.

The practical and presently preferred embodiments of the present invention are illustrative as demonstrated in the following Examples.

However, it will be appreciated that those skilled in the art, on consideration of this disclosure, may make modifications and improvements within the spirit and scope of the present invention.

### EXAMPLES

### Example 1

1. Blood collection: draw 3 ml peripheral blood into a 10-ml syringe through an 18-G needle from the donor by a medical phlebotomist, transfer blood from syringe to sterile 15-ml polystyrene conical centrifuge tube containing 15-30 IU heparin,
2. Peripheral blood mononuclear cell isolation: dilute blood with RPMI1640 to 6 ml total volume, add 4 ml Ficoll separating solution to a 15-ml polystyrene conical centrifuge tube, gently pipette 6 ml diluted blood above Ficoll separating solution avoiding any mixture, spin 20 min at 500×g, room temperature, start with slowest acceleration and no brake, transfer white blood cell-containing band to a new 15-ml polystyrene conical centrifuge tube using sterile pipette, add 4 ml phosphate buffered saline (PBS) containing 0.1% bovine serum albumin (BSA) to 15-ml polystyrene conical centrifuge tube for subsequent washes, during first wash, spin 10 min at 500×g, room temperature, discard the supernatant, resuspend the pellet in 6 ml PBS containing 0.1% BSA, repeat the washes twice more, resuspend the pellet in 3 ml PBS containing 0.1% BSA, count the cells,
3. Cell stimulation: seed 6 wells of 24-well plate with 0.4 ml cell suspension, add cells of three wells with 0.1 ml/well influenza A virus HKX31 strain at a multiplicity of infection 10 per cell as stimulation samples and cells of the other three wells with 0.1 ml/well PBS containing 0.1% BSA as control samples, incubate the plate at room temperature for 1 hour with intermittent rocking of the plate,
4. Cell culture: add 1 ml RPMI1640 containing 10% fetal bovine serum into each wells, culture cells for 0, 1, or 4 hours at 37° C. in a 5% CO2 incubator,
5. Cell staining: collect cells to 5-ml polystyrene round bottom test tube at each time point, add 2 ml PBS containing 0.1% BSA to each tube, spin 5 min at 500×g, room temperature, discard the supernatant, add 50 μl pretitred PerCP-Cy5.5-labeled anti-human CD14 in PBS containing 0.1% BSA to each tube, mix gently, Incubate at 4° C. for 30 min, add 2 ml PBS containing 0.1% BSA to each tube, spin 5 min at 500×g, room temperature, discard the supernatant, fix all samples at 4° C. for 30 min with 0.35 ml 1% paraformaldehyde in PBS, add 2 ml PBS containing 0.03% saponin to each tube, spin 5 min at 500×g, room temperature, discard the supernatant, add 50 μl pretitred PE-Cy7-labled anti-human TNF in PBS containing 0.3% saponin to each tube, mix gently, incubate at 4° C. for 30 min, add 2 ml PBS containing 0.03% saponin to each tube, spin 5 min at 500×g, room temperature, discard the supernatant, resuspend the pellet in 0.5 ml 1% paraformaldehyde in PBS,
6. Sample data acquiring: run samples with optimized photomultiplier settings on a BD LSR II flow cytometer, compensate correctly, acquire the sample data in BD FACSDiva software,
7. Sample data analysis: export the data files in the Flow Cytometry Standard format with an .fcs file extension, load the data into FlowJo, produce detailed dot plot graphical reports, assess the percentage of TNF-producing CD14⁺ monocytes among total CD14⁺ monocytes,

8. Data for basic physiological parameter index: P₀₀=percentage of TNF-producing CD14⁺ monocytes among total CD14⁺ monocytes in uninfected sample at 0 hour post infection, P₁₀=percentage of TNF-producing CD14⁺ monocytes among total CD14⁺ monocytes in influenza infected sample at 0 hour post infection, P₀₁=percentage of TNF-producing CD14⁺ monocytes among total CD14⁺ monocytes in uninfected sample at 1 hour post infection, P₁₁=percentage of TNF-producing CD14⁺ monocytes among total CD14⁺ monocytes in influenza infected sample at 1 hour post infection, P₀₂=percentage of TNF-producing CD14⁺ monocytes among total CD14⁺ monocytes in uninfected sample at 4 hour post infection, P₁₁=percentage of TNF-producing CD14⁺ monocytes among total CD14⁺ monocytes in influenza infected sample at 4 hour post infection,
9. Data for general physiological index: general physiological index=P₀₁/P_{O2}, stimulatory general physiological index=P₁₁/P₁₂, relative general physiological index=P₀₂/P₁₂,
10. Data comparison with the record of physiological parameters in the database,
11. Data presentation and report,
12. Maintain the database for data report.

1. A system and method based on blood components for estimating human general physiological parameters comprises determining the predetermined value of general physiological parameters from basic physiological parameters indicated as the percentage of a particular cytokine-producing cells in a type of blood cells in a human peripheral blood sample without in vitro culture and with two-stage in vitro cultures after being taken from the subject.

2. The particular cytokine of claim 1 wherein said a particular cytokine is tumor necrosis factor (TNF).

3. The type of blood cells of claim 1 wherein said a type of blood cells is monocyte.

4. The two-stage of claim 1 wherein said two-stage is 1 hour and 4 hours.

5. The percentage of claim 1 wherein said the percentage of a particular cytokine-producing cells in a type of blood is the percentage of TNF-producing monocytes in total of monocytes.

6. The basic physiological parameters of claim 1 wherein said basic physiological parameters are the percentage of TNF-producing monocytes in total of monocytes without in vitro culture, the percentage of TNF-producing monocytes in total of monocytes with 1 hour in vitro culture, and the percentage of TNF-producing monocytes in total of monocytes with 4 hours in vitro culture.

7. The value of general physiological parameters of claim 1 wherein said the predetermined value of general physiological parameters from basic physiological parameters is calculated by dividing the percentage of TNF-producing monocytes in total of monocytes with 1 hour in vitro culture of claim 6 by the percentage of TNF-producing monocytes in total of monocytes with 4 hours in vitro culture of claim 6.

8. A system and method based on blood components for estimating human stimulatory general physiological parameters comprises determining the predetermined value of stimulatory general physiological parameters from basic physiological parameters indicated as the percentage of a particular cytokine-producing cells in a type of blood cells following microbial stimulation in a human peripheral blood sample without in vitro culture and with two-stage in vitro cultures after being taken from the subject.

9. The particular cytokine of claim 8 wherein said a particular cytokine is TNF.

10. The type of blood cells of claim 8 wherein said a type of blood cells is monocyte.

11. The two-stage of claim 8 wherein said two-stage is 1 hour and 4 hours.

12. The microbial stimulation of claim 8 wherein said microbial stimulation is influenza A virus at a multiplicity of infection 10 per cell.

13. The percentage of claim 8 wherein said the percentage of a particular cytokine-producing cells in a type of blood is the percentage of TNF-producing monocytes in total of monocytes.

14. The basic physiological parameters of claim 8 wherein said basic physiological parameters are the percentage of TNF-producing monocytes in total of monocytes following influenza A virus at a multiplicity of infection 10 per cell without in vitro culture, the percentage of TNF-producing monocytes in total of monocytes following influenza A virus at a multiplicity of infection 10 per cell with 1 hour in vitro culture, and the percentage of TNF-producing monocytes in total of monocytes following influenza A virus at a multiplicity of infection 10 per cell with 4 hours in vitro culture.

15. The value of stimulatory general physiological parameters of claim 8 wherein said the predetermined value of stimulatory general physiological parameters from basic physiological parameters is calculated by dividing the percentage of TNF-producing monocytes in total of monocytes following influenza A virus at a multiplicity of infection 10 per cell with 1 hour in vitro culture of claim 14 by the percentage of TNF-producing monocytes in total of monocytes following influenza A virus at a multiplicity of infection 10 per cell with 4 hours in vitro culture of claim 14.

16. A system and method based on blood components for estimating human relative general physiological parameters comprises determining the predetermined value of relative general physiological parameters from basic physiological parameters indicated as the percentage of a particular cytokine-producing cells in a type of blood cells following either no or microbial stimulation in a human peripheral blood sample without in vitro culture and with two-stage in vitro cultures after being taken from the subject.

17. The particular cytokine of claim 16 wherein said a particular cytokine is TNF.

18. The type of blood cells of claim 16 wherein said a type of blood cells is monocyte.

19. The two-stage of claim 16 wherein said two-stage is 1 hour and 4 hours.

20. The microbial stimulation of claim 16 wherein said microbial stimulation is influenza A virus at a multiplicity of infection 10 per cell.

21. The percentage of claim 16 wherein said the percentage of a particular cytokine-producing cells in a type of blood is the percentage of TNF-producing monocytes in total of monocytes.

22. The basic physiological parameters of claim 16 wherein said basic physiological parameters are the percentage of TNF-producing monocytes in total of monocytes following no stimulation without in vitro culture, the percentage of TNF-producing monocytes in total of monocytes following influenza A virus at a multiplicity of infection 10 per cell without in vitro culture, the percentage of TNF-producing monocytes in total of monocytes following no stimulation with 1 hour in vitro culture, the percentage of TNF-producing monocytes in total of monocytes following influenza A virus at a multiplicity of infection 10 per cell with 1 hour in vitro culture, the percentage of TNF-producing monocytes in total of monocytes following no stimulation with 4 hours in vitro culture, and the percentage of TNF-producing monocytes in total of monocytes following influenza A virus at a multiplicity of infection 10 per cell with 4 hours in vitro culture.

23. The value of relative general physiological parameters of claim 16 wherein said the predetermined value of relative general physiological parameters from basic physiological parameters is calculated by dividing the percentage of TNF-producing monocytes in total of monocytes following no stimulation with 4 hours in vitro culture of claim 22 by the percentage of TNF-producing monocytes in total of monocytes following influenza A virus at a multiplicity of infection 10 per cell with 4 hours in vitro culture of claim 22.

24. A system and method based on blood components for estimating human physiological parameters comprises 10-ml syringe with an 18-G needle for blood collection, 15-ml polystyrene conical centrifuge tube and 5-ml polystyrene round bottom test tube for preparing, handling and storing blood cells, CO2 incubator for blood cell culture, hemocytometer and light microscope for counting blood cell number, BD LSR II flow cytometer for counting and examining a particular cytokine-producing cells in a type of blood cells, data acquisition network terminal composing of computers for acquiring sample data from BD LSR II flow cytometer, storing sample data, exporting sample data in the Flow Cytometry Standard format with an .fcs file extension and sending exported data to blood analysis server with a secure and agreed format through intranet or internet, and blood analysis server composing of computer database server for analyzing exported data from data acquisition network terminal with a secure and agreed format through intranet or internet, generating human physiological parameters, comparing physiological parameter record of the subject over time, presenting data report to the subject with physical and electronic documents and maintaining the database of the subject.
